(19) United States
(12) Patent Application Publication
(10) Pub. No.: US 2026/0166282 A1
(43) Pub. Date: Jun. 18, 2026

(54) TUBULAR MEMBER FOR AN INTRALUMINAL MEDICAL DEVICE

(71) Applicant: Artiria Medical SA, Borex (CH)

(72) Inventors: Benoît DUBATH, Geneva (CH); Hadrien MICHAUD, Geneva (CH); Guillaume PETIT-PIERRE, Veyrier (CH)

(73) Assignee: Artiria Medical SA, Borex (CH)

(21) Appl. No.: 18/980,668

(22) Filed: Dec. 13, 2024

(51) Int. Cl. A61M 25/09

(52) U.S. Cl. CPC A61M 25/09; A61M 2025/09108; A61M 2025/0915; A61M 2025/09175; A61M 2205/0216; A61M 2207/10

(57) ABSTRACT

A tubular member (101) for a guidewire (201) comprising a distal bending section (2) having distal cut-outs (21) shaped and sized to achieve a distal deflection behaviour, a proximal bending section (4) having proximal cut-outs (41) shaped and sized to achieve a proximal deflection behaviour, and an intermediate bending section (3) between the distal bending section (2) and proximal bending section (4) having intermediate cut-outs (31, 32). The distal and proximal cut-outs (21, 41) are shaped and sized such that the distal bending section (2) is more flexibly deflectable in at least one direction away from a longitudinal axis (L) of the tubular member (101) than the proximal bending section (4). The intermediate cut-outs (31, 32) are shaped and sized such that a first and a second intermediate deflection behaviour differ from each other at at least two different longitudinal positions of the intermediate bending section (3).

The invention relates to a tubular member for an intraluminal medical device, in particular a guidewire, comprising a distal, a proximal, and an intermediate bending section, a guidewire comprising the tubular member, and a manufacturing method of the tubular member according to the independent claims.

Intraluminal, in particular endovascular, medical devices, such as catheters and guidewires, need to be configured to be advanced along tortuous trajectories during medical procedures. Traditionally, guidewires are typically formed by tubular structures such as a coiled tubular structure and/or a hypotube in combination with an inner structure, such as an inner flattened wire. A combination of these structures may enable a transition between different bending stiffnesses of the intraluminal/endovascular medical device. Often a unidirectional anisotropic bending stiffness is required at the distal tip of the device for navigation while a proximal section should only be deflectable to conform to the intraluminal/endovascular path.

Recently, it was established that intraluminal medical devices may be designed to have particular physical characteristics, in particular a specific bending stiffness or a flexural rigidity.

U.S. Pat. No. 8,551,021 B2 discloses a guidewire with an improved flexural rigidity profile which can be achieved by comprising a plurality of slots formed therein such that two different flexural rigidities. A transition between the flexural rigidities may be provided by a polynomial function.

The guidewire of the prior art lacks structural and functional compatibility with actuation mechanisms such as pull wires/push rods which may facilitate deflections.

The prior art further lacks a tubular member having a proximal and distal bending section with different bending behaviours, in particular direction dependent deflectability, which enables reliable actuation without buckling or plastic deformations.

In the prior art, buckling of the intraluminal medical instrument, e.g. a guidewire, due to non-elastic deformations may also occur when the medical instrument is advanced distally in a lumen, e.g. a vasculature, while assuming a deflected shape by interaction with the anatomy.

The prior art further lacks a simple, cheap, and reliable tubular member to be used for an intraluminal medical device, in particular a guidewire, which enables an optimized transition of deflection behaviour for intraluminal medical applications. In particular, the prior art lacks a robust and compact tubular member which enables reliable miniaturization and sufficient structural integrity for narrow endovascular application while enabling localized bending at a distal tip section(s).

In particular, it was found that even a small mismatch in stiffness between a non-isotropic tip and the preceding quasi-isotropic proximal section of the intraluminal medical device can lead to a local catastrophic buckling. This may lead to adverse patient outcomes, prolonged surgical time, reduced efficiency and efficacy of the intervention and even elevated morbidity rates.

It is the object of the present invention to overcome these and other disadvantages of the prior art. The present invention shall provide a tubular member for a guidewire, a guidewire comprising the tubular member, and a method of manufacturing the tubular member which solve the above-mentioned technical problems and enable a reliable bending behaviour of a distal bending section of the tubular member in intraluminal medical procedures.

The tubular member for a guidewire according to the invention comprises a distal bending section having distal cut-outs shaped and sized to achieve a distal deflection behaviour, a proximal bending section having proximal cut-outs shaped and sized to achieve a proximal deflection behaviour, and an intermediate bending section between the distal bending section and proximal bending section having intermediate cut-outs. The distal and proximal cut-outs are shaped and sized such that the distal bending section is more flexibly deflectable in at least one direction, in particular exactly one direction, away from a longitudinal axis of the tubular member than the proximal bending section. The intermediate cut-outs are shaped and sized such that a first and a second intermediate deflection behaviour differ from each other at at least two different longitudinal positions of the intermediate bending section.

The deflection behaviour, in particular the distal deflection behaviour, preferably the distal and intermediate deflection behaviour, may include a direction-dependent stiffness of the respective bending section. The proximal deflection behaviour may have a non-direction dependent stiffness.

The distal and/or proximal deflection behaviour may be essentially constant along a longitudinal direction of the tubular member, i.e. non-changing on a macroscopic level from a distal to a proximal end of the section.

The proximal/intermediate/distal deflection behaviour, e.g. an anisotropic or quasi-isotropic deflection behaviour, may be essentially uniform when the respective section is viewed as a whole, i.e. deflection over a macroscopic level. However, on a microscopic level, i.e. over short longitudinal subsections of the respective sections, the according cut-outs may lead to localized variations/non-uniform deflection behaviours of the respective sections. The terms anisotropic, in particular unidirectional anisotropic, and isotropic bending stiffness of a section refer to the macroscopic level, i.e. over the respective bending section as a whole, irrespective of localized variations on the microscopic level.

The macroscopic level can relate to lengths of more than 10 times an outer diameter of the tubular member, e.g. more than 3 mm. The microscopic level can relate to lengths less than 1 time the outer diameter, e.g. less than 0.3 mm.

The “longitudinal axis” is a curvilinear axis of the tubular member which follows a curved and/or non-linear path corresponding to the shape of the tubular member rather than a straight line.

The terms “proximal” and “distal” are defined from a clinician's perspective and not from an anatomical perspective, i.e. the term “distal” relates to a direction further away from the clinician and “proximal” further towards the clinician.

The at least one of distal, proximal, and intermediate cut-outs may extend entirely through the tubular member from one outer circumferential surface to an opposite inner surface of the tubular member, i.e. extend through a wall of the tubular member.

This enables to provide a tubular member which is deflectable safely, reliably, and repeatably, preferably in one deflection plane or more than one deflection plane, by an inner actuation element which is adapted for force transmission, in particular a pull wire or push rod.

The tubular member may be a monolithic tubular member, in particular a monolithic tubular member devoid of a coiled member.

The tubular member may comprise or consists of polymers, metals, metal alloys or metal oxides. In particular the tubular member may comprise or consist of polyamide, polyimide, poly ethylene (PE), poly propylene (PP), polyether etherketone (PEEK), acrylonitrile butadiene styrene (ABS), epoxys, polytetrafluoroethylene (PTFE), polyurethane, thermoplastic polyurethanes (TPU), polyamide, Polyether block amide (PeBax), Kevlar, carbon fiber reinforced polymer, glass fiber-reinforced polymer, metal braiding-reinforced polymer, titanium, stainless steel, nickel titanium alloy (nitinol), nickel-chromium alloy, cobalt alloy, tungsten, cobalt, chrome, nickel, aluminum, copper, molybdenum or any combination thereof.

The monolithic tubular member enables improved structural integrity of the tubular member by eliminating weak points prone to failure under stress, optimized, manufacturing, and a simpler design of a guidewire with fewer components, in particular movable components.

In a preferred embodiment, the tubular member comprises nitinol, or similar shape memory alloys or polymers, due to its superelastic properties. Stainless steel may be used as a cheaper alternative.

The tubular member may have a cross-sectional profile which has a shape and/or size which varies along the longitudinal axis of the tubular member. In particular the cross-sectional size may decrease from a proximal end to a distal end of the tubular member. The tubular member may comprise or consist of at least one cross-sectional profile selected from a circular, ellipsoidal, oval, or irregular cross-sectional profile.

The intermediate deflection behaviour at a distal end of the intermediate bending section may be substantially similar to or the same as the distal deflection behaviour. Additionally or alternatively, the intermediate deflection behaviour at a proximal end of the intermediate bending section may be substantially similar to or the same as the proximal deflection behaviour. The intermediate cut-outs may be shaped and sized such that the intermediate deflection behaviour gradually or essentially continuously transitions from the distal end to the proximal end.

This enables to reduce load peaks and material deformations and/or prevents buckling of sections of the tubular member upon deflection such that overstressing can be prevented. Thereby, the risk of material failure can be reduced while repeatability of the deflectability is ensured.

The intermediate deflection behaviour at a distal end may be the first intermediate deflection behaviour. The intermediate deflection behaviour at a proximal end may be the second intermediate deflection behaviour, i.e. these are different from each other. The first deflection behaviour at the distal end may be unidirectionally deflectable in at least one, in particular exactly one direction, and has a higher degree of deflection than the second deflection behaviour at the proximal end when subjected to the same force. The intermediate bending section may be deflectable in this manner by exerting customary forces on the tubular member, in particular by longitudinally compressing or extending the tubular member, e.g. via the inner actuation element.

The distal deflection behaviour may include an anisotropic stiffness of the distal bending section, in particular a unidirectional anisotropic stiffness, with respect to lateral deflections of the distal bending section away from the longitudinal axis of the tubular member.

An anisotropic stiffness of the bending section(s) means having different bending stiffnesses dependent on the directions of deflection away from the longitudinal axis (L) of the tubular member.

This anisotropic, preferably unidirectional anisotropic, stiffness, enables the distal bending section to be used for navigation in tortuous body ducts reliably. In a preferred embodiment, the anisotropic stiffness of the distal deflection behaviour is larger or at most equal to than any of the other anisotropic stiffnesses of the proximal and intermediate deflection behaviour.

The term unidirectional anisotropic stiffness of the bending section(s) is defined as a higher or lower stiffness when force is applied in exactly one deflection direction away from the longitudinal axis but the other of the higher or lower stiffness when deflected in any other deflection direction away from the longitudinal axis. The deflection in a deflection plane may encompass a deflection away from the longitudinal axis to a deflected shape, and a reverse deflection to a straight shape or opposingly deflected shape in the same plane.

The proximal deflection behaviour may have an essentially quasi-isotropic stiffness with respect to lateral deflections from the longitudinal deflections from the longitudinal axis of the tubular member.

This essentially quasi-isotropic stiffness may enable the proximal bending section to deflect in order to be introduced into tortuous paths of bodily lumens, in particular of a vasculature of a patient. This may also provide sufficient structural integrity of the proximal bending section to not be deflected to the same degree as the distal bending section when the tubular member is subject to a longitudinally axial force.

The term quasi-isotropic flexibility refers to the property of the tubular member to deflect nearly the same in all directions away from the longitudinal axis. A direction-dependent variation in deflection may be less than 258, in particular less than 15%, preferably less than 108, between any two radial directions under identical customary load conditions.

At least one of the proximal, distal, and intermediate cut-outs, in particular at least the proximal and intermediate cut-outs, may be arranged in sets of one, of two, of three, or of four along a circumference of the tubular member. The cut-outs of the set may be circumferentially separated by material bridges of the tubular member.

These sizes of sets of cut-outs enable sufficient tunability of the deflection behaviour of the sections while providing sufficient structural integrity, even for miniaturized dimensions, without unduly complicating the manufacturing process.

The material bridges of the tubular member may all have the same or similar shape/size/circumferential dimension such that the mechanical forces during deflection are distributed more evenly across the entire sections to avoid material fatigue or failure.

At least two distal cut-outs, in particular a majority of the distal cut-outs, preferably all of the distal cut-outs, of the distal set, longitudinally neighbouring each other may be arranged on at least one common axis parallel to the longitudinal axis of the tubular member, in particular such that the at least one common axis leads through at least one circumferential midpoint of the at least two cut-outs. In addition or alternatively, the largest distal cut-out of at least one distal set of distal cut-outs is at least 100%, in particular at least 150%, preferably at least 200%, larger than a circumferential length of the shortest distal cut-out. Alternatively, the at least one distal set of the distal bending section may comprise only one distal cut-out.

The term parallel to the longitudinal axis means parallel to a tangential line of the longitudinal axis at the respective location. If the longitudinal axis is deflected, i.e. extends along a curved and/or non-linear path, an element parallel to the longitudinal axis may also assume a curved/non-linear path. An axis parallel to the longitudinal axis, e.g. a common axis leading through circumferential midpoints, may therefore also be a curvilinear axis which follows a curved and/or non-linear path.

The circumferential midpoint means a point located equidistant from both circumferential ends of the cut-out. When the tubular member's surface is conceptualized as an unfolded two-dimensional pattern, the circumferential midpoint corresponds to a geometric center of the cut-out in the flattened projection.

The term “neighbouring” sets of cut-outs is defined as sets which are not separated by more than one or two sets of cut-outs along the longitudinal direction. The term “directly neighbouring” sets of cut-outs refers to neighbouring sets of cut-outs which are not separated by any sets of cut-outs.

Arranging at least two distal cut-outs on a common axis simplifies the manufacturing process and provides a distal bending section which has an anisotropic deflection behaviour, in particular a unidirectional anisotropic deflection behaviour.

A plurality of directly longitudinally neighbouring distal cut-outs, i.e. without being separated by any sets of cut-outs, or all cut-outs of the directly longitudinally neighbouring distal sets respectively, may be arranged on the common axis/common axes. The plurality of directly longitudinally neighbouring distal sets may each comprise two, more than two, more than three, in particular more than four, optionally more than five, distal cut-outs. The distal cut-outs of the distal set(s) may be distributed in a circumferential direction of the tubular member, in particular equidistantly, i.e. at equal angular intervals along the circumference, and/or with material bridges having the same circumferential length.

Distal sets of cut-outs comprising two or one cut-out may enable particularly beneficial structural integrity due to fewer but circumferentially longer material bridges.

A first common axis parallel to the longitudinal axis may lead through first circumferential midpoints of at least two distal cut-outs of a first type. A second common axis parallel to the longitudinal axis may lead through second circumferential midpoints of at least two distal cut-outs of a second type. A third, fourth, fifth, and/or sixth common axis parallel to the longitudinal axis may lead through a third, fourth, fifth, and/or sixth circumferential midpoints of at least two distal cut-outs of a third, fourth, fifth, and/or sixth type.

A majority or all of the distal cut-outs may be arranged on the first and the second common axis; the first, the second, and the third common axis; the first, the second, the third, and the fourth common axis; the first to fifth common axis; or the first to sixth common axis.

At least two sets of proximal cut-outs longitudinally neighbouring each other may be circumferentially offset with respect to each other by a circumferential displacement, optionally selected from a range of more than 5%, in particular more than 15%, preferably more than 22.5%, with respect to the circumference of the tubular element. The circumferential lengths of the proximal cut-outs of at least one set may be essentially equal to each other with any variation in a circumferential length being less than 5%.

This enables to provide a proximal bending section having a more isotropic bending flexibility, in particular an essentially quasi-isotropic bending flexibility. The circumferential displacement of the proximal cut-outs longitudinally neighbouring each other may be 25% with respect to the tubular member, in particular with any variation in a circumferential displacement being less than 5%.

At least three sets of intermediate cut-outs longitudinally neighbouring each other may be circumferentially offset with respect to each other. The first and second set of the three sets may be circumferentially offset by a first circumferential length and the second and third set of the three sets may be circumferentially offset by a second circumferential length. The first circumferential length may be different than the second circumferential length. The at least three, in particular more than three, longitudinally neighbouring sets may be all arranged offset by a circumferential length in the same circumferential direction to form a helical pattern.

The offset of the longitudinally neighbouring intermediate cut-outs may be an offset from the circumferential midpoint of the respective cut-outs.

The sets being circumferentially offset with respect to each other may relate to each cut-out of the respective offset set to be offset by the same circumferential offset as the other cut-outs of the set.

Different circumferential offsets of the intermediate cut-outs enable a variable intermediate deflection behaviour, in particular a variable intermediate deflection behaviour which is variably dependent on the deflection direction and a longitudinal position of the intermediate bending section.

Arranging the neighbouring sets to form the helical pattern may provide an intermediate bending section which has a variable intermediate deflection behaviour which is dependent on the deflection direction, such that the deflection behaviour varies along a longitudinal axis in a helical pattern.

The intermediate bending section may comprise at least three additional neighbouring sets which are also circumferentially offset as previously described in the same circumferential direction to form an additional helical pattern. The helical patterns may be arranged opposingly to each other, e.g. one helical pattern may be sinistral helical pattern, and the other helical pattern may be a dextral helical pattern.

The proximal most longitudinally neighbouring set of the helical pattern of the at least three longitudinally neighbouring sets which circumferential midpoints are offset by at least one circumferential length in the same circumferential direction may have an offset with respect to a distal most longitudinal neighbouring set. The offset may be selected from a range of 60° to 120°, in particular 80° to 100°, preferably 85° to 95° or

$$90 ° / \frac{π}{2} .$$

The proximal most longitudinally neighbouring set may be arranged at the proximal end of the intermediate bending section and the distal most longitudinally neighbouring set may be arranged on the distal end of the intermediate bending section. The tubular member may comprise at least two helical patterns, in particular at least one sinistral and at least one dextral helical pattern, having the previously described proximal most and distal most neighbouring set respectively.

The at least one helical pattern may may have a constant pitch or a varying pitch along the longitudinal direction of the intermediate bending section. The varying pitch may have a linearly, quadratically, or exponentially varying pitch. These constant or varying pitches may enable to tune the bending behaviour to specific procedural indications for intravascular procedures.

At least one of the neighbouring sets of intermediate cut-outs, in particular a plurality of neighbouring sets of intermediate cut-outs, of the intermediate bending section may be separated by at least one separating intermediate cut-out, in particular a plurality of separating intermediate cut-outs of a separating intermediate set which has at least one circumferential midpoint. The circumferential midpoint of the separating intermediate cut-out(s) may be essentially arranged on at least one common axis parallel to the longitudinal axis of the tubular member which leads through at least one circumferential midpoint of at least one distal cut-out of the distal set, in particular through the majority of distal cut-outs of the distal set, of the distal bending section. Any circumferential variation of the at least one circumferential midpoint with respect to the common axis may be less than 15%, in particular less than 10%, preferably less than 5%, of the circumference of the tubular member.

Such separating intermediate cut-outs having circumferential midpoints which are on the common axis of the at least one distal cut-out enable the intermediate bending section to have an anisotropic stiffness, in particular a unidirectional anisotropic stiffness, similar to the anisotropic stiffness of the distal bending section.

The tubular member may have separating intermediate sets which are not arranged on a common axis parallel to the longitudinal axis. These separating intermediate sets may instead enable a more isotropic bending behaviour.

A circumferential length of a plurality of separating intermediate cut-outs distributed along the intermediate bending section along the longitudinal axis may, at least on average, decrease from the distal to the proximal end of the intermediate bending section. Thereby, the anisotropic stiffness, in particular a unidirectional anisotropic stiffness, of the intermediate bending section may decrease from the distal to the proximal end of the intermediate bending section. This may provide a gradually or, from a macroscopic level, quasi-continuous transition from the distal to the proximal end of the intermediate bending section and, therefore, e.g. prevent rapid stiffness variations which may lead to load peaks and enhance durability and improve safety when using the tubular member for medical intraluminal applications.

The intermediate bending section may comprise at least one pair of symmetric sets of intermediate cut-outs which are different from each other. Each symmetric set has intermediate cut-outs with circumferential midpoints which are equispaced around the circumference of the tubular member.

Equispaced circumferential midpoints may be arranged along the circumference such that a variation from a completely uniform midpoint distribution does not exceed ±5°.

The symmetric sets may be arranged such that the circumferential midpoints of the same symmetric set are 180°, 120°, 90°, 72°, or 60° circumferentially offset to each other with respect to a circumferential midpoint of the tubular member.

This may enable an equal force distribution on either lateral side of the intermediate bending section for two, three, four, five, or six cut-outs per symmetric set.

In a preferred embodiment, the intermediate bending section comprises sets, in particular symmetric sets, of cut-outs with only two cut-outs which simplifies miniaturization while retaining a sufficient material strength of the separating material bridges.

The tubular member may comprise a plurality of pairs of symmetrical sets. At least one pair may comprise intermediate cut-outs which have the same circumferential lengths respectively. The at least one pair of symmetric sets, in particular a plurality of pairs of symmetric sets, may have different circumferential lengths of intermediate cut-outs with respect to each other.

A plurality of neighbouring symmetric sets, in particular directly neighbouring symmetric sets, may have cut-outs which are circumferentially offset with respect to each other in the same manner.

A first plurality of neighbouring symmetric sets may have cut-outs which are circumferentially offset with respect to each other in a first circumferential direction in a helical pattern. A second plurality of neighbouring symmetric sets may have cut-outs which are circumferentially offset with respect to each other in a second circumferential direction opposed to the first circumferential direction in a helical pattern.

The intermediate sets of intermediate cut-outs may each comprise a first intermediate cut-out and a second intermediate cut-out. The first intermediate cut-out of a plurality of neighbouring intermediate sets may sequentially decrease in circumferential length from the distal end to the proximal end. In addition, the second intermediate cut-out of the plurality of neighbouring sets may sequentially increase in circumferential length from the distal end to the proximal end, preferably until the first and second circumferential length are essentially equal to each other.

This provides a gradual or essentially continuously transition of the deflection behaviour along the intermediate bending section from an anisotropic deflection behaviour of the distal bending section to an essentially quasi-isotropic deflection behaviour of the proximal bending section. This further enables an even force distribution and enhances structural integrity such that the distal bending section can be securely deflected unidirectionally while the intermediate bending section unidirectionally deflects most at a distal end, gradually less in a proximal direction, and least at a proximal end.

In addition or alternatively, the intermediate sets or some of the intermediate sets of cut-outs may only comprise one cut-out.

The first intermediate cut-out of the plurality of neighbouring intermediate sets sequentially decreasing in circumferential length from the distal end to the proximal end may no longer be present at a location between the distal and proximal end. The intermediate bending section may only comprise intermediate sets comprising or consisting of the second cut-out from this location to the proximal end.

The distal bending section and/or the intermediate bending section may have a length selected from a range of 5 mm to 50 mm, in particular 5 mm to 30 mm, preferably 12 mm to 17 mm.

The intermediate bending section may be shorter than the distal bending section. The intermediate bending section may be selected from a range of 5 mm to 10 mm. The distal bending section may be selected from a range of 10 mm to 15 mm.

These lengths provide a sufficient compromise of deflectability of the sections and structural integrity in view of intraluminal, in particular vascular applications, preferably neurovascular applications which necessitate navigation in narrow and tortuous vasculatures. In particular, an intermediate bending section of this length enables a reliable transition between the bending stiffness of the distal and proximal bending section to reduce a risk of buckling and enabling elastic deformations of the tubular member.

The proximal bending section of the tubular member may comprise an actuation region, in particular an elastic actuation region. The previously described elastic actuation region may comprise or consist of cut-outs which are sized and shaped such that two sections of the actuation region are movable in opposing direction towards or away from each other to compress or extend a longitudinal length of the elastic actuation region.

Another aspect of the present invention relates to a guidewire comprising the previously described tubular member. The guidewire may comprise an elongated actuation element, in particular a pull wire, for actuating at least one of the bending sections, in particular at least the distal bending section, preferably at least the distal bending section and the intermediate bending section.

The elongated actuation element may be configured for exerting an axial force on the tubular member, in particular a tensile and/or compressive force. The elongated actuation element may be a push-rod.

The pull wire may be attached to a distal tip of the guidewire such that applying the tensile force, in particular by retracting a pull wire or tensioning it indirectly by compressing/extending the elastic actuation region, may result in a deflection of the guidewire.

The actuation element may be attached to a proximal end of the guidewire, in particular proximally from the actuation region, in particular the elastic actuation region, of the tubular member. Thereby, the actuation element may be actuated by applying the axial force by compressing/extending the elastic actuation region.

The elastic actuation region may be formed by a coiled member or by a tubular section comprising cut-outs. The elastic actuation region formed by the tubular section may be a subsection of the previously described tubular member at a proximal end of the tubular member.

Another aspect of the present invention relates to a powered steerable system comprising the previously described guidewire including the tubular member. The powered steerable system may comprise an actuation unit configured to be coupled to the actuation element and actuate the actuation element. The powered steerable system may comprise an input interface for receiving an input, in particular a user input, specifying a deflection state of the guidewire. The powered steerable system may comprise a control unit coupled to the user interface. The control unit may be configured to generate a control command based on the input which may be transmitted via an output interface to the actuation unit. The actuation unit may be configured to deform the guidewire to the deflection state of the guidewire based on the control command.

Another aspect of the present invention relates to a method of manufacturing a tubular member for a guidewire as previously described. The method comprises locally modifying a tubular member by removing material of the tubular member to form a plurality of cut-outs. The material is removed by at least one of (i) photonic material removal, (ii) micro-dicing, (iii) electrical discharge machining, (iv) electrochemical machining, (v) chemical machining, (vi) ultrasonic machining, (vii) water jet cutting, and (viii) abrasive blasting to achieve the distal, proximal, and intermediate deflection behaviour of the distal, proximal, and intermediate bending section.

The invention is now described with reference to certain embodiments and figures which show:

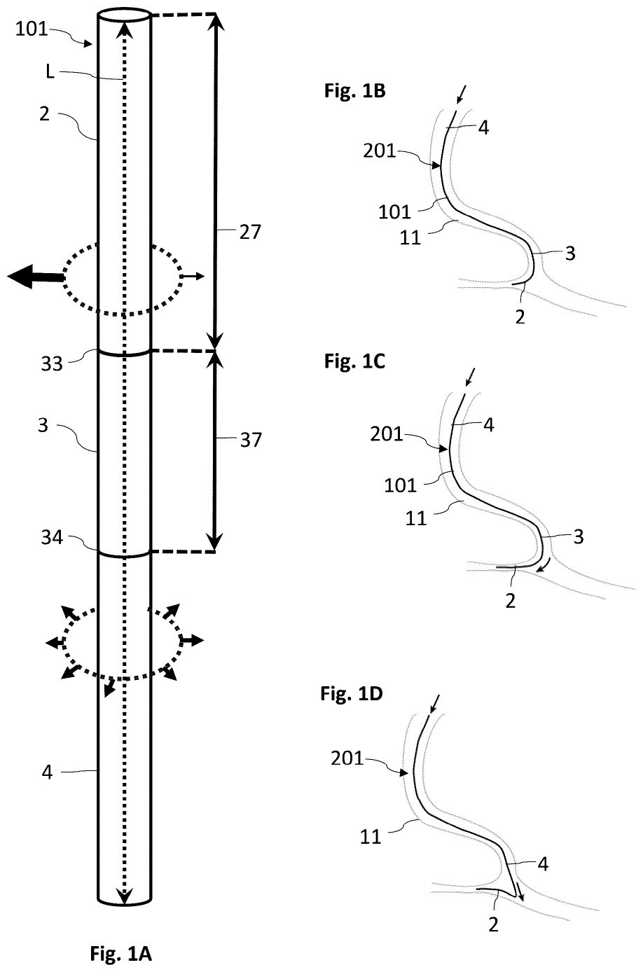

FIG. 1A: a perspective view of a tubular member according to the invention comprising a proximal, intermediate, and distal bending section,

FIGS. 1B-1C: a guidewire comprising the tubular member according to the invention being advanced along a tortuous vasculature of a patient,

FIG. 1D: a guidewire comprising a tubular member comprising no intermediate bending section which buckles when deflected to navigate through the tortuous vasculature,

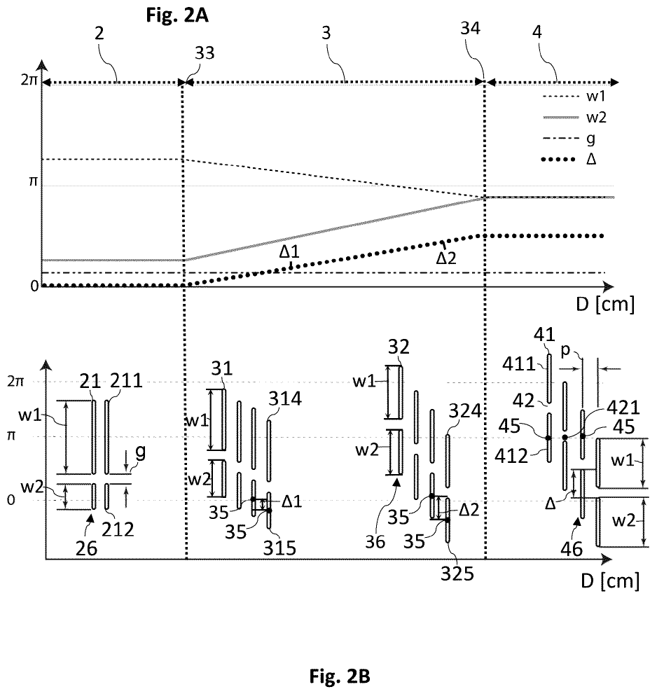

FIG. 2A: a graphical representation of circumferential lengths of first and second cut-outs, a circumferential gap between the cut-outs, and a circumferential offset of the cut-outs with respect to a distance from a distal tip of the distal bending section of an embodiment of the tubular member,

FIG. 2B: the cut-outs of the distal, intermediate, and proximal bending section of the tubular member according to FIG. 2A,

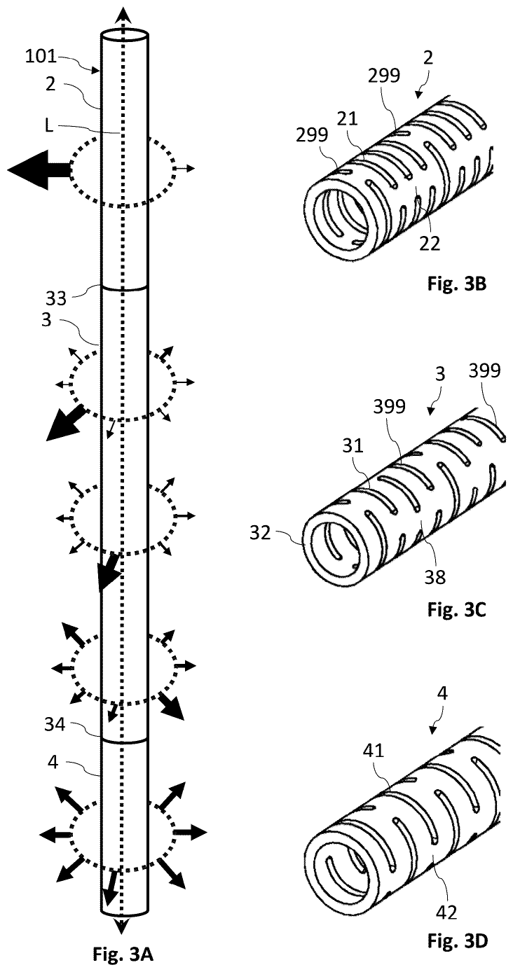

FIG. 3A: a perspective view of a tubular member according to an embodiment of the invention comprising an intermediate bending section having an intermediate deflection behaviour which gradually decreases in a helical pattern from a distal end to a proximal end of the intermediate bending section,

FIGS. 3B-3D: a perspective view of the distal bending section, the intermediate bending section, and the proximal bending section and their respective cut-outs,

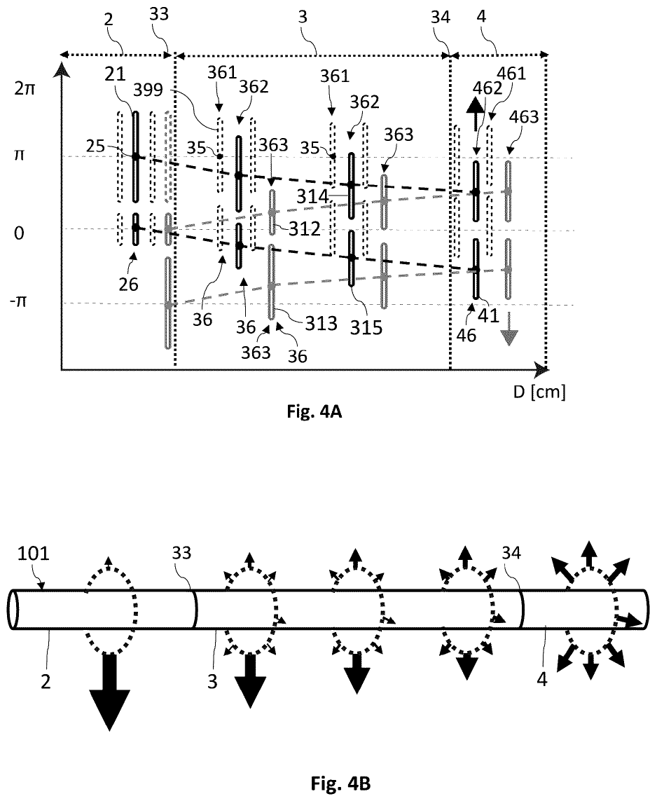

FIG. 4A: a graphical representation of cut-outs of the distal, intermediate, and proximal bending section of the tubular member according to an alternative embodiment of the invention along a longitudinal axis,

FIG. 4B: a perspective view of a tubular member according to an alternative embodiment of the invention according to FIG. 4A,

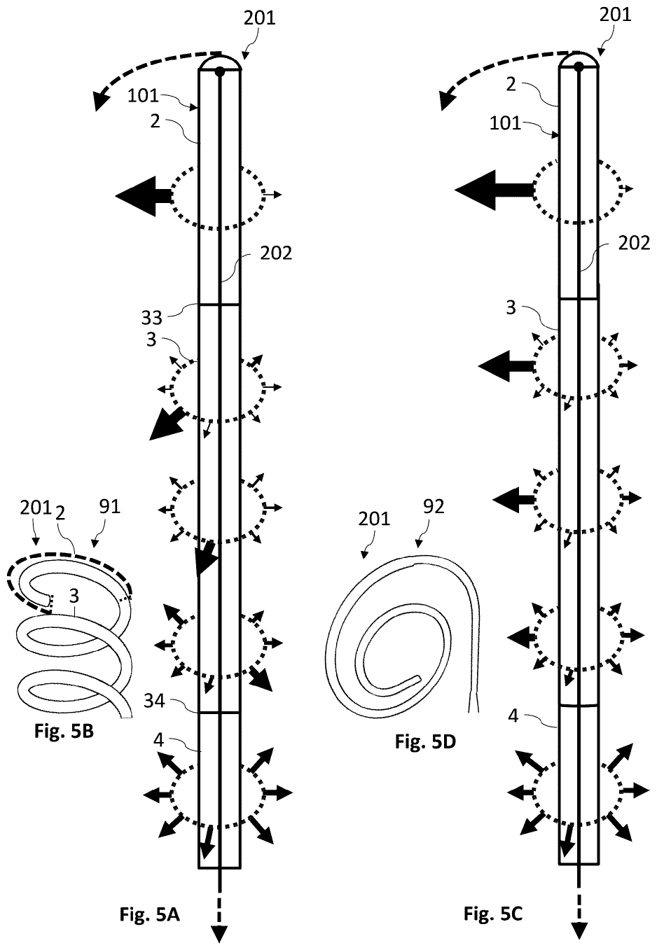

FIGS. 5A and 5B: a perspective view of a guidewire comprising the tubular member according to FIG. 3A and an actuation element, and the guidewire being deflected to a helical shape,

FIGS. 5C and 5D: a perspective view of a guidewire comprising the tubular member according to FIG. 4B and an actuation element, and the guidewire being deflected to a coiled shape,

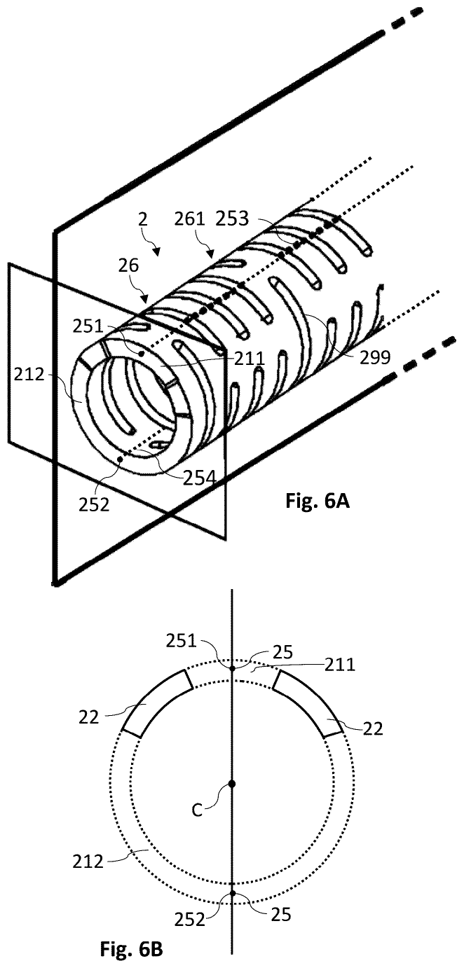

FIG. 6A: a perspective view of a distal bending section of a tubular member according to an embodiment of the invention, and

FIG. 6B: a cross-sectional view of the distal bending section of the tubular member according to an embodiment of the distal bending section according to FIG. 6A.

FIG. 1A shows a perspective view of a tubular member 101 according to the invention comprising a proximal bending section 4, an intermediate bending section 3, and a distal bending section 2. The distal bending section 2 has a length 27 of 12.6 mm. The intermediate bending section 3 has a length 37 of 6 mm. Both these bending sections 2, 3 may also have a similar length, in particular 15 mm. These lengths enable the tubular member 101 to be manoeuvrable during intravascular neurovascular applications. The overall length of the bending sections 2, 3, 4 may be selected from typical lengths for neurovascular applications, in particular between 160 cm to 315 cm, preferably 200 cm. The tubular member 101 of FIG. 1A has an essentially circular shape and is made of nitinol with a hydrophilic and/or hydrophobic coating. The distal bending section 2 may comprise a hydrophobic coating and the proximal bending section a hydrophilic coating. The intermediate bending section 3 may comprise a hydrophobic or hydrophilic coating, in particular a coating gradually transitioning from hydrophobic to hydrophilic in the proximal direction. All the bending sections have a deflection behaviour which is defined by an amount, an orientation, and/or an alignment of proximal, distal, and intermediate cut-outs in the tubular member 101.

FIG. 1A shows that the distal bending section 2 has an essentially unidirectional anisotropic stiffness at a macroscopic level. The distal bending section 2 is deflectable away from a longitudinal axis L of the tubular member 101 in one direction more easily than in any other directions away from the longitudinal axis L when exerting longitudinal tensile forces on the tubular member 101. This deflection behaviour is denoted in FIG. 1A by a big arrow pointing laterally away from the longitudinal axis in the distal bending section 2. In an opposing direction of deflection, a smaller arrow points laterally away from the longitudinal axis, indicating that the distal bending section 2 is also deflectable, at least to a degree, in an opposing direction, i.e. in a plane spanned by the unidirectional deflection and the longitudinal axis.

The proximal bending section 4 of the tubular member 101 has an essentially quasi-isotropic bending stiffness at a macroscopic level, i.e. is essentially equally deflectable in any direction when exerting tensile forces on the tubular member 101. This enables the bending section 4 to be deflectable enough to deflect accordingly to a vasculature of the patient without being deflectable to a similar degree as the distal bending section when the longitudinal tensile force is applied to the tubular member by an actuation element.

The intermediate bending section 3 of the tubular member 101 is configured to transition from the distal deflection behaviour of the distal bending section 2 to the proximal deflection behaviour of the proximal bending section 4. This necessitates at least two different intermediate deflection behaviours of the intermediate bending section 3, in particular a gradual or essentially continuous transition of the intermediate deflection behaviour. In a preferred embodiment, the deflection behaviour of the intermediated bending section 3 at a distal end 33 corresponds to the unidirectional anisotropic deflection behaviour of the distal bending section 2. The deflection behaviour of the intermediate bending section 3 at a proximal end 34 may instead correspond to the deflection behaviour of the proximal bending section 4. The deflection behaviour of the intermediate bending section 3 will be described in more detail in FIGS. 3A, 4B, 5A, and 5C.

FIGS. 1B and 1C show a guidewire 201 comprising the tubular member 101 according to the invention being advanced along a tortuous vasculature 11 of a patient. Due to an intermediate bending section 3 (see FIG. 1A), the bending stiffness of the tubular member 101 transitions from the anisotropic regime of the distal bending section 2 to the quasi-isotropic regime of the proximal bending section 4 gradually or continuously on a macroscopic level. This enables the guidewire 201, when actuated by an actuation element such as a pull wire, to minimize load peaks in the tubular member 101. This enables repeatable and safe deflection of the guidewire 201 without buckling in the transition region, i.e. the intermediate bending section.

FIG. 1D shows a guidewire 201 comprising a tubular member comprising no intermediate bending section between a proximal and distal bending section 4, 2 being advanced in the vasculature of a patient.

The guidewire 201 of FIG. 1D may buckle upon deflection by an actuation element such as a pull wire compressing the tubular member at the intersection between the anisotropic distal bending section 2 and the quasi-isotropic proximal bending section 4 due to the abrupt change in deflection behaviour.

The guidewire 201 of FIG. 1D may also buckle due to being advanced while being introduced into a branch of the vasculature 11 based on interactions with the anatomy of the patient. The force for distally advancing the guidewire 201 can lead to an increased tension on the deflected region, in particular the transition between the distal section 2 and the proximal section 4, due to the distal section 2 being confined by the tortuous path of the vasculature 11. Due to a lack of an intermediate section which gradually distributes this tension, a localized tension peak between the distal and proximal section 2, 4 may lead to buckling and/or non-elastic deformations of the guidewire 201.

FIG. 2A shows a graphical representation of circumferential lengths w1, w2 of cut-outs 21, 31, 32, 41, a circumferential gap g between the cut-outs 21, 31, 32, 41, and a circumferential offset Δ of the cut-outs 21, 31, 32, 41 with respect to a distance from a distal tip of the distal bending section of an embodiment of the tubular member. The y-axis of FIG. 2A is designated in radians and a value denotes the radians arc over which the respective parameter extends over the circumferential length of the tubular member. A gap g between the distal, intermediate, and proximal cut-outs 211, 212; 314, 315; 324, 325; 411, 412 of all bending sections 2, 3, 4 always has the same circumferential length as well.

FIG. 2B shows the cut-outs 21, 31, 32, 41 of the distal bending section 2, the intermediate bending section 3, and the proximal bending section 4 according to FIG. 2A.

The distal bending section 2 of FIGS. 2A and 2B has cut-outs 211, 212 which have two different cut-outs 21 of different circumferential lengths w1, w2. FIGS. 2A and 2B show a second shorter cut-out 212 with the length w2 and a first larger cut-out 211 with a length w1. The circumferential length w1 of the first larger cut-out 211 is more than 200% larger than a circumferential length of the circumferential length w2 of the shorter circumferential length of the shorter cut-out 212. The first and second cut-outs 211, 212 are arranged circumferentially adjacent on the tubular member such that their circumferential midpoints are 180°/n apart. These first and second cut-outs 211, 212 form a set 26 of two cut-outs 211, 212. The cut-outs 211, 212 do not change in position, orientation or circumferential length along the longitudinal axis of the distal bending section 2, i.e. the circumferential offset Δ between the distal cut-outs 21 is essentially zero. This arrangement of cut-outs 211, 212 enables the distal bending section 2 to be unidirectionally and anisotropically deflectable laterally away from a longitudinal axis of the tubular member in the direction of the circumferentially larger cut-out 211.

The intermediate bending section 3 comprises a plurality of intermediate cut-outs 31, 32. The intermediate cut-outs 31, 32 both have a first cut-out 314, 324 and a second cut-outs 315, 325. The first cut-outs 314, 324 have a circumferential length w1 which gradually decreases along a distance from a distal end 33 of the intermediate bending section 3 to a proximal end 34 of the intermediate bending section 3. The second cut-outs 315, 325 have a circumferential length w2 gradually increases along the distance opposingly and at the same rate as the decrease in circumferential length w1 of the first distal cut-outs 314, 325. The gradual decrease and increase in circumferential length w1, w2 is only schematically shown in FIG. 2B for different cut-outs 31, 32 of the intermediate bending section 3, i.e. only two different circumferential lengths w1, w2 of these cut-outs 314, 324; 315, 325 are displayed.

The circumferential offset Δ between the first and second intermediate cut-outs 314, 315; 324, 325 sequentially increases from the distal end 33 to the proximal end 34 of the intermediate bending section 3. The circumferential offsets A between the cut-outs 314, 315; 324, 325 are all arranged in the same circumferential direction to from a helical pattern. The circumferential offset Δ in FIGS. 2A and 2B is defined as the distance of a circumferential midpoint 35 between two directly neighbouring intermediate cut-outs 314, 315; 324, 325. A circumferential offset Δ1 of intermediate cut-outs 314, 315 of a more distal set 36 is smaller than a circumferential offset Δ2 of intermediate cut-outs 324, 325 of a more proximal set 36. As shown by FIG. 2A, the offset Δ may essentially sequentially increase such that the offset Δ at a distal end 33 is zero and at a proximal end 34 corresponds to the offset of cut-outs 41 of the proximal bending section 4. This may lead to an essentially gradual or essentially continuous transition on a macroscopic level of the intermediate deflection behaviour of the intermediate bending section 3 from the distal end 33 to the proximal end 34.

The proximal bending section 4 comprises sets 46 of proximal cut-outs 41. Each set has two cut-outs 411, 412 which are equal in circumferential length w1, w2. The circumferential offset Δ is selected such that the respective circumferential midpoints 45 of the cut-outs 411, 412 of a set are not arranged on a common axis with a circumferential midpoint 421 of a material bridge 42 of the gap g of a directly neighbouring set 46 of cut-outs 411, 412. The cut-outs 411, 412 of neighbouring sets have an offset Δ of 85° in a circumferential direction. Thereby, the cut-outs 411, 412 of neighbouring sets 46 are arranged along a helical pattern along the longitudinal axis of the tubular member to avoid defining specific bending directions. This enables the proximal bending section 4 to exhibit a quasi-isotropic bending behaviour.

The previously described embodiments of the distal, intermediate, and proximal bending sections of FIGS. 2A and 2B may be combined with other any of the one or two distal, intermediate, and/or proximal bending sections of other embodiments.

A longitudinal interdistance p between directly neighbouring sets 26, 36, 46 of the bending sections 2, 3, 4 in FIGS. 2A and 2B is always constant for illustrative purposes. However, the interdistance p may also be chosen in accordance to achieve a certain bending stiffness of the bending section 2, 3, 4, e.g. a smaller interdistance p may be chosen for the distal bending section to further facilitate deflection upon exertion of a longitudinal tensile force on the tubular member and a larger interdistance p between directly neighbouring sets 26, 36, 46 may be chosen for the proximal bending section 4 to reduce the amount of deflection upon exertion of the longitudinal tensile force.

FIG. 3A shows a perspective view of a tubular member 101 according to an embodiment of the invention comprising an intermediate bending section 3 having an intermediate deflection behaviour which gradually decreases in a helical pattern from a distal end 33 to a proximal end 34 of the intermediate bending section 3.

The distal bending section 2 has cut-outs to achieve a uniform anisotropic stiffness, such that a lateral deflection from a longitudinal axis L of the tubular member 101 in one plane can be achieved upon an exertion of a tensile force on the tubular member 101.

The intermediate bending section 3 has cut-outs which are arranged in the previously described helical pattern by offsets arranged in the same circumferential direction (see FIGS. 2A and 2B). The circumferential length of the intermediate cut-outs also changes from one circumferential length of a cut-out of a set being much larger than the other one at the distal end 33 to both cut-outs of an intermediate set having essentially the same circumferential length at a proximal end 34.

In this manner, the intermediate deflection behaviour gradually changes from the distal end 33 to the proximal end 34. At the distal end 33, the intermediate deflection behaviour essentially corresponds to a unidirectional anisotropic distal deflection behaviour but at the proximal end, the intermediate deflection behaviour essentially corresponds to a quasi-isotropic proximal deflection behaviour.

The anisotropic deflection behaviour is caused by the different circumferential lengths of the cut-outs of sets and uniform arrangement without any offset of longitudinally neighbouring sets at the distal bending section and at the distal end 33 of the intermediate bending section.

The quasi-isotropic deflection behaviour is caused by the cut-outs having the same circumferential length and being equally distributed along the proximal bending section 4/proximal end of the intermediate bending section 3, e.g. by alternatingly arranging the circumferential midpoints of the cut-outs and bridges as previously described in FIGS. 2A and 2B.

Due to the one directional offsets between the cut-outs of neighbouring sets (see FIGS. 2A and 2B), the unidirectional deflectability of the intermediate section 3 of FIG. 3A also assumes a helical pattern which describes half a helix from the distal to the proximal end 33, 34, i.e. 180°/π. Concurrently, the deflection behaviour changes from the unidirectional anisotropic to the quasi-isotropic intermediate deflection behaviour.

FIGS. 3B to 3D show a perspective view of a section of the distal bending section 2, the intermediate bending section 3, and the proximal bending section 4 and their respective cut-outs 21, 31, 41. The cut-outs 21, 31, 41 of the bending sections 2, 3, 4 and the material bridges 22, 38, 42 separating cut-outs of sets 26, 36, 46 (see FIG. 4A) are arranged similarly as previously described in FIG. 4A. However, additional separating cut-outs 299, 399 are provided between every three directly neighbouring sets of cut-outs 21, 31 of the distal and intermediate bending section 2, 3. These separating cut-outs 299 of the distal bending section 2 may enable a little more isotropic bending behaviour for the otherwise unidirectional anisotropic distal bending section 2. The separating cut-outs 399 of the intermediate bending section 3 may be arranged correspondingly to the cut-outs 21 of the distal bending section 2, e.g. such that circumferential midpoints of the separating cut-outs 399 are arranged on a common axis of the circumferential midpoints of the largest cut-outs of the distal bending section 2. In this manner, the intermediate bending section 3 may also have a unidirectional deflection behaviour to a certain degree as will be described later in further detail.

FIG. 4A shows a graphical representation of cut-outs 21; 312, 313, 314, 315, 399; 41 of a distal, intermediate, and proximal bending section 2, 3, 4 of the tubular member according to an alternative embodiment of the invention along a longitudinal axis starting at a distal tip of the tubular member. The y-axis of FIG. 4A display an angular range from −2π to 2π, i.e. two entire circumferential turns for illustrative purposes. Some cut-outs 21; 312, 313, 314, 315, 399; 41 are not displayed or not displayed for both ranges −2π to 0 and 0 to 2π for better visibility. All sets 26, 36, 46 of cut-outs of FIG. 4A comprise two cut-outs across the circumference of the tubular member but different embodiments include more, in particular three, four, five, or six cut-outs.

The cut-outs 21 of the distal bending section 2 are essentially arranged as previously described. Each distal set 26 of distal cut-outs 21 is arranged equidistantly to each other and each distal cut-out 21 is arranged parallel to each other. The distal cut-outs 21 all have the same circumferential length as its directly neighbouring cut-out 21 of a neighbouring set 26. The first and second distal cut-outs 21 of a plurality of distal sets 26 each consist of two cut-outs 21 which have circumferential midpoints 25 which are arranged on a common axis. Both larger upper cut-outs 21 and smaller lower cut-outs of longitudinally neighbouring sets are arranged on separate common axes.

The common axes in FIG. 4A are denoted as a dashed grey line passing through π and 0 and are parallel to the longitudinal axis of the tubular member. The upper distal cut-out 21 of the distal bending section 2 in FIG. 4A has a greater circumferential length than a lower cut-out of the distal bending section 2 similar to FIGS. 2A and 2B. This may support the unidirectional anisotropic bending behaviour of the distal bending section 2.

The intermediate bending section 3 comprises a plurality of sets 36 including symmetric sets 362, 363 of intermediate cut-outs 312, 313; 314, 315. The intermediate bending section 3 further comprises separating sets 361 of cut-outs 399. The symmetric sets 362, 363 of the intermediate bending section 3 are longitudinally intermittently arranged with the separating sets 361 of the intermediate bending section 3.

The separating sets 361 comprise two cut-outs 399 respectively. Both of these separating cut-outs 399 of the separating sets 361 have circumferential midpoints 35 which are arranged on a first and second common axis leading through π and 0 which are parallel to the longitudinal axis of the tubular member. Both these common axes lead through the circumferential midpoints 25 of the upper and lower cut-outs 21 of the distal sets 26. Upper intermediate cut-outs 399 of the longitudinally spaced apart separating sets 361 sequentially decreases in circumferential length while the lower intermediate cut-out 399 sequentially increases in circumferential length from a distal end 33 to a proximal end 34 of the intermediate bending section 3. At the distal end 33 of the intermediate bending section 3, the circumferential length of the separating cut-outs 399 essentially correspond to a circumferential length of the cut-outs 21 of the distal bending section 2. At the proximal end 34 of the intermediate bending section 3, the circumferential length of the separating cut-outs 399 essentially correspond to a circumferential length of the cut-outs 41 of the proximal bending section 4. This enables the intermediate bending section 3 to sequentially transition from the distal bending behaviour to the proximal bending behaviour gradually on a macroscopic level.

The symmetric sets 362, 363 comprise two intermediate cut-outs 312, 313; 314, 315 respectively. The intermediate cut-outs 312, 313; 314, 315 of each symmetric set 362, 363 are arranged such that their circumferential midpoints shown as black dots in FIG. 4A are spaced apart a circumferential radial range of π with respect to each other. If the intermediate bending section 3 instead comprises sets 362, 363 of more than two circumferential cut-outs 312, 313; 314, 315, in particular three, four, five, or six cut-outs per set, the circumferential midpoints may be distributed along the circumference equidistantly, e.g. distributed at an angle of 120°, 90°, 72°, or 60° with respect to a cross-sectional circumferential midpoint of the intermediate bending section 3.

First symmetric sets 362 comprise cut-outs 312, 313 which have circumferential midpoints which are sequentially offset along the longitudinal axis from the distal end 33 to the proximal end 34 in the same circumferential direction as indicated by the dashed black line in FIG. 4A. Second symmetric sets 363 comprise cut-outs 314, 315 which have circumferential midpoints which are sequentially offset along the longitudinal axis from the distal end 33 to the proximal end 34 in the same circumferential direction as indicated by the dashed grey line in FIG. 4A. The cut-outs 314, 315 of the second symmetric sets 363 are offset in an opposing direction to the cut-outs 312, 313 of the first symmetric sets 362. Thereby, both symmetric sets 362, 363 can essentially form a sinistral and dextral helical pattern respectively. A pitch of these helical patterns may be constant or increase linearly, quadratically, or exponentially.

FIG. 4A shows that both helical patterns only extend over a circumferential radial range of 90°/π/2 from the distal end 33 to the proximal end 34. Thereby, the unidirectional anisotropic bending behaviour corresponding to the distal bending section 2 can be gradually transitioned to a quasi-isotropic bending behaviour of the proximal bending section 4. In addition, the double helical pattern enables the intermediate bending section to also retain the unidirectional aspect of the distal bending section 2 which steadily decreases along the proximal direction of the intermediate bending section while the isotropic bending behaviour gradually increases until an omnidirectional bending behaviour of the proximal bending section 4 can be achieved.

The proximal bending section 4 comprises a plurality of sets 46. The sets 462, 463 which denote the first sets of the proximal bending section 4, and the ends of the sinistral and dextral helical patterns are essentially arranged on a common axis parallel to the longitudinal axis. The sets 46 may also be arranged as previously described in FIG. 2A to achieve a quasi-isotropic deflection behaviour. Separating sets 461 of the proximal bending section are essentially arranged on the same previously described common axis of the upper largest cut-outs 21 of the distal bending section 2. All circumferential midpoint of the proximal cut-outs 41 of the proximal bending section 4 are spaced apart equally along the circumference and have the same circumferential length.

FIG. 4B shows a perspective view of a tubular member 101 according to an alternative embodiment of the invention according to FIG. 4A. The tubular member 101 comprises an intermediate bending section 3 having an intermediate deflection behaviour which gradually decreases from a distal end 33 to a proximal end 34 of the intermediate bending section 3 while remaining essentially unidirectionally deflectable with a concurrently smaller degree along the longitudinal axis. The opposing helical patterns of the cut-outs of the intermediate bending section 3 enable to convert the unidirectional anisotropic deflectability from the distal bending section 2 reliably to a quasi-isotropic deflectability of a proximal bending section 4.

FIGS. 5A and 5B show a perspective view of a guidewire 201 comprising the tubular member 101 according to FIG. 3A and an actuation element 202, and the guidewire 201 being deflected to a helical shape 91. The actuation element 202 is adapted for applying a longitudinal tensile force on the tubular member 101. The actuation element 202 of FIGS. 5A and 5B is formed by a pull wire 202 which is attached to a distal tip of the distal bending section 2 which has an atraumatic rounded tip. By retracting/tensioning the pull wire 202, the distal and intermediate bending section 2, 3 can be deflected to the helical shape 91 shown in FIG. 5B.

The distal bending section 2 shown in FIG. 5B is deflected essentially in a single plane while the intermediate bending section 3 extends longitudinally in a three-dimensional helical shape. This may be achieved by the previously described single helical pattern of cut-outs of the intermediate bending section 3, e.g. as described in detail in FIGS. 2A and 2B. The helical pattern of the deflection behaviour concerning the declining unidirectional deflectability of the intermediate bending section 3 extends over half a helix from a distal end 33 to a proximal end 34 of the intermediate bending section 3. Thereby, a gradual transition between the unidirectional anisotropic distal bending behaviour of the distal bending section 2 to the quasi-isotropic proximal bending behaviour of a proximal bending section 4 can be established.

FIGS. 5C and 5D show a perspective view of a guidewire 201 comprising the tubular member 101 according to FIG. 4B and an actuation element 202, and the guidewire 201 being deflected to a coiled shape 92. The guidewire 201 is similarly formed as described having regard to FIGS. 5A and 5B but includes an intermediate bending section 3 which has a double helical pattern of cut-outs with separating cut-outs as described in FIGS. 4A and 4B. Thereby, the coiled shape 92 is essentially merely achieved by the distal and intermediate bending section 2, 3 being deflected in a single plane, i.e. the distal and intermediate bending section are deflectable laterally away from a longitudinal axis of the guidewire 201 in the same plane. The unidirectional deflectability of the intermediate bending section 3 decreases continuously on a macroscopic level from a distal end 33 where the deflectability corresponds to the deflectability of the distal bending section 2 to a proximal end 34 where the deflectability essentially correspond to the proximal bending section 4.

Depending on the procedural indication of an intraluminal medical procedure a guidewire 201 may be selected from either of the two designs described in FIGS. 5A and 5B or FIGS. 5C and 5D.

FIG. 6A shows a perspective view of a distal bending section 2 of a tubular member according to an embodiment of the invention. Every three directly neighbouring sets 26 of cut-outs 211, 212 an intermittent separating set 261 of cut-outs 299 is arranged. The plurality of three directly neighbouring sets 26 each comprise two cut-outs 211, 212, a first cut-out 211 which has a small circumferential length and a second cut-out 212 which has a larger circumferential length. The plurality of first cut-outs 211 each have a first circumferential midpoint 251 and the plurality of second cut-outs 212 each have a second circumferential midpoint 252. The first circumferential midpoints 251 are arranged on a first common axis 253 which extends parallel to the longitudinal axis of the tubular member. The second circumferential midpoints 252 are arranged on a second common axis 254 which extends parallel to the longitudinal axis as well. The first and second common axis 253, 254 are circumferentially spaced apart by 180°/π with respect to a central longitudinal axis of the distal bending section 2. Thereby, the distal bending section 2 is deflectable in a symmetry plane leading through both the first and second common axes 253, 254 which is included in FIG. 6A as a rectangular shape. The distal bending section 2 has a unidirectional and anisotropic bending stiffness which enables a lateral deflection away from a longitudinal axis in the direction of the second cut-outs 252 having a larger circumferential length.

FIG. 6B shows a cross-sectional view of the distal bending section 2 according to the embodiment of the distal bending section 2 according to FIG. 6A, corresponding to the cross-section depicted in FIG. 6A as a rectangular shape. FIG. 6B shows the cut-outs 211, 212 of the sets 26 of the distal bending section 2. The majority of the cut-outs 211, 212 are arranged as shown in FIG. 6B. Only a single separating set 261 of cut-outs 299 is arranged between every three directly neighbouring sets 26 of cut-outs 211, 212.

FIG. 6B shows that the cut-outs 211, 212 of the sets 26 have a circumferential midpoint 25 which lies on the first and second common axis 251, 252 respectively. The cut-outs 211, 212 are separated by material bridges 22 which both have the same circumferential length.

FIG. 6B further shows that a connecting line denoting an intersection of the symmetry plane of FIG. 6A with the common axes 251, 252 leads through a geometric cross-sectional center C of the distal bending section, i.e. they are circumferentially spaced apart from each other 180°/π on the circumference of the distal bending section 2 with respect to the cross-sectional center C.

1. A tubular member for an intraluminal medical device comprising:
A distal bending section having distal cut-outs shaped and sized to achieve a distal deflection behaviour,
A proximal bending section having proximal cut-outs shaped and sized to achieve a proximal deflection behaviour, and
An intermediate bending section between the distal bending section and proximal bending section having intermediate cut-outs,
wherein the distal and proximal cut-outs are shaped and sized such that the distal bending section is more flexibly deflectable in at least one direction away from a longitudinal axis of the tubular member than the proximal bending section, wherein the intermediate cut-outs are shaped and sized such that a first and a second intermediate deflection behaviour differ from each other at at least two different longitudinal positions of the intermediate bending section.

2. The tubular member according to claim 1, wherein the tubular member is a monolithic tubular member.

3. The tubular member according to claim 1, wherein the intermediate deflection behaviour is at least one of (i) substantially similar to or the same as the distal deflection behaviour at a distal end of the intermediate bending section and (ii) substantially similar to or the same as the proximal deflection behaviour at a proximal end of the intermediate bending section.

4. The tubular member according to claim 1, wherein the distal deflection behaviour includes an anisotropic stiffness of the distal bending section with respect to lateral deflections of the distal bending section away from the longitudinal axis of the tubular member.

5. The tubular member according to claim 1, wherein the proximal deflection behaviour has an essentially quasi-isotropic stiffness with respect to lateral deflections from the longitudinal axis of the tubular member.

6. The tubular member according to claim 1, wherein at least one of the proximal, distal, and intermediate cut-outs are arranged in sets of one, of two, of three, or of four along a circumference of the tubular member and the cut-outs of the set are circumferentially separated by material bridges of the tubular member.

7. The tubular member according to claim 6, wherein the tubular member comprises at least one of:
(i) at least two distal cut-outs of the distal set, longitudinally neighbouring each other which are arranged on at least one common axis parallel to the longitudinal axis of the tubular member,
(ii) the largest distal cut-out of at least one distal set of distal cut-outs which is one of: at least 100%, at least 150%, and at least 200% larger than a circumferential length of the shortest distal cut-out, and
the at least one distal set of the distal bending section comprises only one distal cut-out.

8. The tubular member according to claim 6, wherein the tubular member comprises at least one of:
(i) at least two sets of proximal cut-outs longitudinally neighbouring each other which are circumferentially offset with respect to each other by a circumferential displacement and
(ii) the circumferential lengths of the proximal cut-outs of at least one set which are essentially equal to each other with any variation in a circumferential length being less than 5%.

9. The tubular member according to claim 6, wherein
At least three sets of intermediate cut-outs longitudinally neighbouring each other are circumferentially offset with respect to each other, wherein the first and second set of the three sets are circumferentially offset by a first circumferential length and the second and third set of the three sets are circumferentially offset by a second circumferential length, wherein the first circumferential length is different than the second circumferential length.

10. The tubular member according to claim 6, wherein at least one of the neighbouring sets of intermediate cut-outs of the intermediate bending section are separated by at least one separating intermediate cut-out of a separating intermediate set, which has at least one circumferential midpoint which is essentially arranged on at least one common axis parallel to the longitudinal axis of the tubular member which leads through at least one circumferential midpoint of at least one distal cut-out of the distal set of the distal bending section with any circumferential variation of the circumferential midpoint with respect to the common axis being at least one of: less than 15%, less than 10%, and less than 5%, of the circumference of the tubular member.

11. The tubular member according to claim 6, wherein the intermediate bending section comprises at least one pair of symmetric sets of intermediate cut-outs which are different from each other, and wherein each symmetric set has intermediate cut-outs with circumferential midpoints which are equispaced around the circumference of the tubular member.

12. The tubular member according to claim 6, wherein the intermediate sets of intermediate cut-outs each comprise a first intermediate cut-out and a second intermediate cut-out, and wherein the first intermediate cut-out of a plurality of neighbouring intermediate sets sequentially decreases in circumferential length from the distal end to the proximal end.

13. The tubular member according to claim 1, wherein at least one of the distal bending section and the intermediate bending section have a length selected from one of a range of 5 mm to 50 mm, 5 mm to 30 mm, and 12 mm to 17 mm.

14. A guidewire comprising the tubular member according to claim 1, wherein the guidewire comprises an elongated actuation element for actuating at least one of the bending sections.

15. A method of manufacturing a tubular member for an intraluminal medical device according to claim 1, comprising:
locally modifying a tubular member by removing material of the tubular member to form a plurality of cut-outs by at least one of
(i) photonic material removal,
(ii) micro-dicing,
(iii) electrical discharge machining,
(iv) electrochemical machining,
(v) chemical machining,
(vi) ultrasonic machining,
(vii) water jet cutting, and
(viii) abrasive blasting
to achieve the distal, proximal, and intermediate deflection behaviour of the distal, proximal, and intermediate bending section.

16. The guidewire according to claim 3, wherein the intermediate cut-outs are shaped and sized such that the intermediate deflection behaviour gradually or essentially continuously transitions from the distal end to the proximal end.

17. The tubular member according to claim 4, wherein the anisotropic stiffness of the distal bending section is a unidirectional anisotropic stiffness.

18. The tubular member according to claim 7, wherein the at least one common axis leads through at least one circumferential midpoint of the at least two distal cut-outs.

19. The tubular member according to claim 8, wherein the circumferential offset is selected from a range of one of: more than 5%, more than 15%, and more than 22.5% with respect to the circumference of the tubular element.

20. The tubular member according to claim 9, wherein the at least three longitudinally neighbouring sets are all arranged offset by the circumferential length in the same circumferential direction to form a helical pattern.

21. The tubular member according to claim 12, wherein the second intermediate cut-out of the plurality of neighbouring sets sequentially increases in circumferential length from the distal end to the proximal end.
